(19) United States
(12) Patent Application Publication
(10) Pub. No.: US 2017/0258457 A1
(43) Pub. Date: Sep. 14, 2017

(54) Dunn Biopsy: A Method of Fine Needle Biopsy of Mammalian Tissue Over Time as a Diagnostic Tool

(71) Applicant: Clemson University Research Foundation, Clemson, SC (US)

(72) Inventors: Heather W. Dunn, Clemson, SC (US); Kathryn A. Elliott, Clemson, SC (US); Thomas R. Scott, Clemson, SC (US); Matthew Burns, Clemson, SC (US)

(21) Appl. No.: 15/352,108

(22) Filed: Nov. 15, 2016

Related U.S. Application Data
(60) Provisional application No. 62/256,416, filed on Nov. 17, 2015.

(51) Int. Cl. A61B 10/02; C12Q 1/68

(52) U.S. Cl. CPC A61B 10/0233; C12Q 1/6806; A61B 2503/40; A61B 2010/0208

(57) ABSTRACT

A method for correlation attributes of mature mammals tissue to pre-pubescent mammals is provided. The method comprises providing a biopsy needle with a bore diameter of at least 0.9081 mm (20 gauge) to less than 2.413 mm (13 gauge) with a specimen notch, of at least 2 mm to no more than 16 mm; inserting the needle into said mammalian tissue of a pre-pubescent living mammal and extracting a sample tissue.

### CROSS-REFERENCE TO RELATED APPLICATIONS

This application claims priority to pending U.S. Provisional Patent Application No. 62/256,416 filed Nov. 17, 2015 which is incorporated herein by reference.

### BACKGROUND

The present invention is related to an improved method for analysis of pre-pubescent mammalian tissue as a basis for determination of eventual pathology or for improved prediction of at least one attribute of animals such as birds, fish or mammals. More specifically, the present invention is related to a method of analysis of prepubescent tissue for subsequent comparison to mature tissue, to a phenotype of the animal at maturity, a blood sample, or diseased tissue thereby leading to a diagnostic, or predictive, tool for determination of expected mammalian attributes at maturity.

There are many fields of study and commerce wherein a mammalian characteristic is of utmost importance. Some characteristics are detrimental to the mammal; such as cancers, tumors, diseases in addition to others; and others may be a positive attribute; such as breeding characteristics, meat production or quality characteristics, lactation characteristics in addition to others. Many of these characteristics manifest at maturity, or at least post-pubescence, which is long after much expense has occurred in raising the mammal. Cancer diagnosis, for example, is often initiated based on symptomatic mammals thereby severely limiting the options for mitigation. In production mammals, such as those intended for meat production, the attributes may manifest after the mammal has been raised to maturity thereby hindering effective segregation of breed stock from production stock.

Testing of prepubescent mammalian tissue has been problematic and therefore infrequently pursued in the past. Biopsy approaches have been unacceptable due to the creation of tissue damage and the risk that the damaged tissue has the potential to bias the data since the test mammal may be appreciably traumatized, at least locally, or differentiation and growth characteristics may be altered. Other techniques have proven to be less reliable.

There has been an ongoing desire for a non-invasive or minimally-invasive method of tissue sampling, preferable in a pre-pubescent mammal, which neither traumatizes nor appreciably alters differentiation of growth of the mammal and which can be repeated at the same location multiple times. Such an advance is provided herein.

### SUMMARY OF THE INVENTION

It is an object of the invention to provide a method of tissue sampling in mammals, preferably pre-pubescent mammals, wherein the sampling does not alter differentiation or growth in the region of sampling.

It is an object of the invention to provide a method for sampling tissue, preferably from pre-pubescent mammals, for comparison with subsequent mammalian attributes thereby providing a mechanism for pre-pubescent determination of attributes in a mature mammal for pathological or predictive evaluation.

These and other advantages, as will be realized, are provided in a method for predicting attributes of mature mammals based on pre-pubescent tissue. The method comprises providing a spring activated biopsy needle with a bore diameter of at least 0.9081 mm (20 gauge) to less than 2.413 mm (13 gauge) with a specimen notch, of at least 2 mm to no more than 16 mm; inserting the needle into tissue of a pre-pubescent mammal and extracting a tissue sample from the mammal.

### BRIEF DESCRIPTION OF FIGURES

FIG. 1 is a micrograph of calf mammary tissue at 2 weeks of age.

FIG. 2 is a micrograph of swine mammary tissue at 2 weeks of age.

FIG. 3 is a micrograph of lamb mammary tissue at 9 weeks of age.

FIGS. 4 and 5 are charts demonstration swine growth rate during sampling.

FIGS. 6-11 are sequential images of swine mammary tissue, from the same animal, at days 4, 8, 11, 18, 25 and 32 respectively.

FIG. 12 is a series of micrographs illustrating the Developmental Maturity Index.

### DESCRIPTION

The inventive technique is specific to a method for sampling mammalian tissue, preferably pre-pubescent mammalian tissue, with minimal adverse immediate effect on the tissue, minimal effect on differentiation of the tissue and minimal effect on growth of the tissue to post-pubescence and maturity. More specifically, the present invention provides a method for correlating pre-pubescent mammalian tissue characteristics to post-pubescent manifested mammalian attributes thereby providing the tools necessary for prevention of disease or optimization of production effectiveness.

The invention will be described with reference to the figures forming an integral, but non-limiting, component of the disclosure.

An integral component of the inventive technique is the use of a conventional spring activated biopsy needle with a bore diameter of at least 0.09081 mm (20 gauge) to less than 2.413 mm (13 gauge), and more preferably at least 1.270 mm (18 gauge) to less than 2.413 mm (13 gauge), modified for a sample length, or specimen notch, of at least 2 mm to no more than 16 mm and more preferably at least 8 mm to no more than 16 mm. A Jorgenson Labs Inc. J0528 with a 14 gauge by 9 cm needle can be modified to adjust the sample size. A sample size above about 2.413 mm is unnecessary since tissue trauma can be detrimental to the mammal. Below a sample size of about 0.9081 mm the cross-sectional size of the sample is insufficient. Above a specimen length of about 16 mm the tissue trauma can be detrimental and below a specimen length of about 2 mm the sample size is insufficient. Sample sizes as described herein have been demonstrated to be sufficient for testing yet minimally invasive to the mammal with no adverse side effects and no attributes correlated to the sampling.

Sampling is preferably done in asymptomatic tissue of a pre-pubescent living mammal. A particular feature of the invention is the ability to obtain a sample which is tissue specific. By obtaining smaller samples, as detailed herein, tissue containing hair, blood and skin can be excluded from the sample. This allows for the sampling of specific tissue such as adipose, skin, muscle or specific combinations thereof while excluding unwanted tissue. While it is preferable to sample asymptomatic tissue in a living pre-pubescent mammal, the sampling is preferably on a mammal at less than 3 months of age, more preferably less than 2 months of age and in some embodiments at less than 1 month of age. Sampling as early as 1 day of age has been demonstrated to be suitable for demonstration of the invention. The sampling is minimally invasive and therefore the same mammal can be sampled repeatedly, such as every week, into and through puberty thereby allowing for tissue analysis of the same animal from pre-pubescence through pubescence for ultimate comparison and correlation with post-pubescent or post-mortem samples or attributes.

More specifically, the sampling is done on a mammal selected from swine, cattle, ovine, caprine, primates, rodent, canine, and feline.

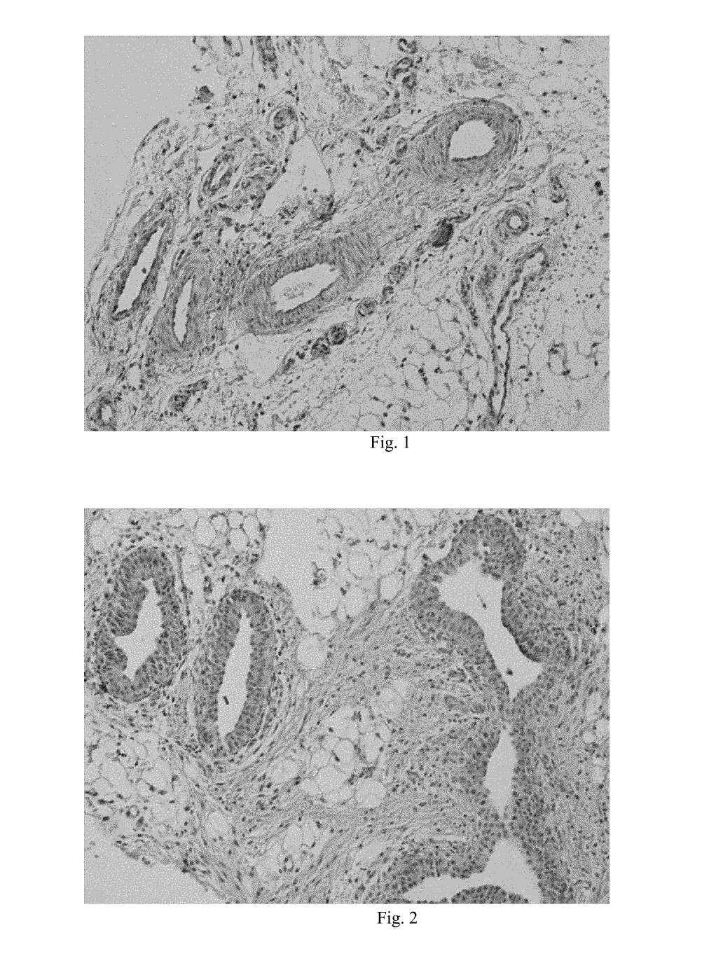

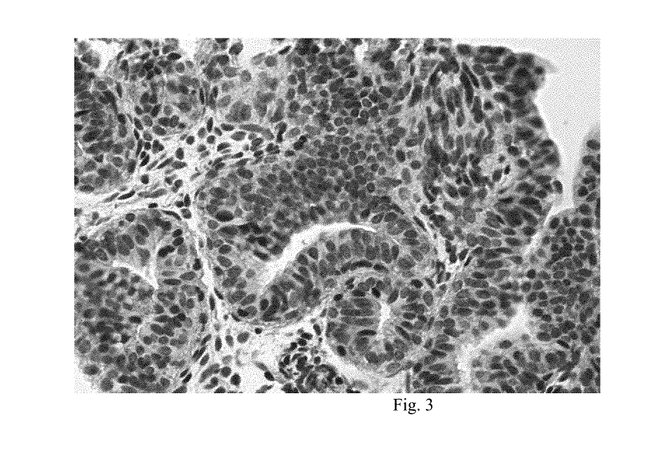

FIGS. 1-3 are cross-sectional images of mammary tissue of different species sampled in accordance with the invention wherein the samples were paraffin embedded, sectioned and stained. The images demonstrate the effectiveness of the technique with regards to the ability to obtain adequate resolution suitable for interspecies differentiation of living pre-pubescent mammals. FIG. 1 is an image of mammary tissue of a calf at 2 weeks of age. FIG. 2 is an image of mammary tissue of swine at 2 weeks of age. FIG. 3 is an image of mammary tissue of a lamb at 9 weeks of age.

For the purposes of the present invention sampling will be artificially separated into two categories for the purposes of discussion even though the sampling technique may, or may not, vary between the two and the sample location may or may not vary. One category is specific to disease detection and the other is specific to attribute detection. Disease detection samples will focus, without limit, on mammalian tissue which is studied for subsequent frequency of formation of disease, particularly cancer. Attribute detection samples will focus, without limit, on mammalian tissue which is studied for the purposes of production quality. The primary difference is that samples focused on disease detection may lead to disease abatement efforts thereby increasing the length and/or quality of life whereas samples focused on attribute detection may lead to efforts to optimize production quality and cost.

For the purposes of clarification sampling of swine, as a representative mammal, will be further described. The individual animal is preferably manually restrained for no longer than about 10 minutes each and the sample site cleaned such as with a triple wash of chlorhexidine and betadine. Blood is preferably collected and a body weight is preferably taken during each sample period for comparison purposes and further study. A numbing agent, such as Lidocaine (2%), is preferably administered at the sample site via intradermal and subcutaneous routes, and a small tissue sample removed, as described herein, from the parenchyma of the mammary gland, for females, or from the rump or neck, for males. In the females, it is preferable to sample different mammary glands, or alternate locations, for successive sampling. Sample collection is preferably obtained with a 14 gauge biopsy needle as described herein, and blood is preferably collected for secondary testing, confirmation and possible correlation. The animal is preferably evaluated for approximately 5 minutes following the procedure to ensure any bleeding has stopped from the biopsy sample site and from the blood collection site. If bleeding is observed pressure is preferably applied. After being released from the restraint the animal preferably returns to a normal diet and housing preferably with daily observation for 3-5 days for observation of any signs of infection. If the animal appears to have an infection appropriate treatment is preferably provided. During this procedure, the animal is preferably closely monitored for any signs of pain.

It is preferable to test swine from multiple litters. In one embodiment, 3 animals per litter with 2 being females and 1 being a male are preferably tested. In one embodiment a female from different litters will be euthanized every 2 or 3 weeks during the tissue collection cycle with euthanization occuring at weeks 4 and 7 in order to validate the technique thereby insuring an adequate sample which is representative of the mammary gland. It is preferable that a repeat sample collection occurs at least 1 day after a previous sample collection. Euthanized animals can be used to measure how deep the mammary tissue expands into the body as the animal grows to compare patterning of a necropsied tissue sample with the biopsy sample and to confirm there is low or no variability in sampling live animals using this technology.

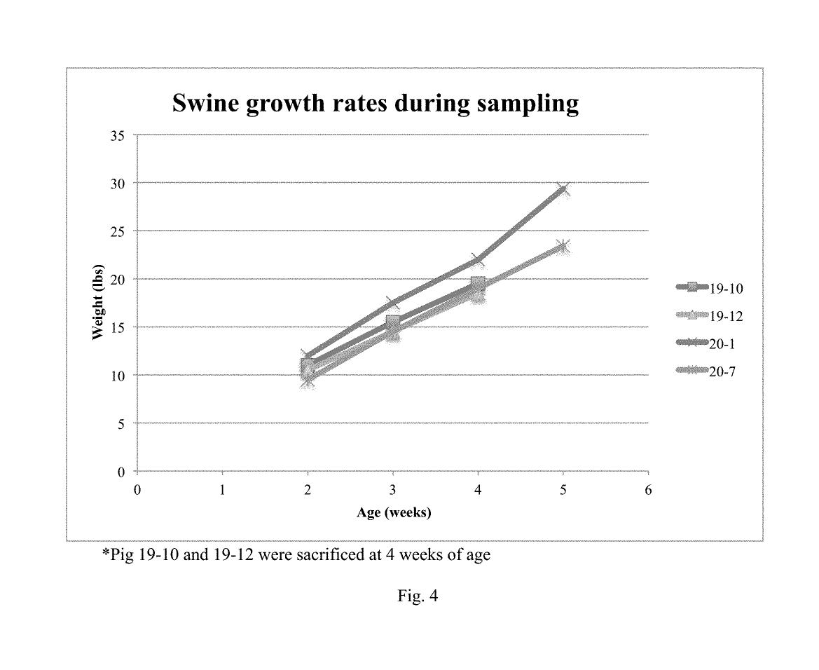

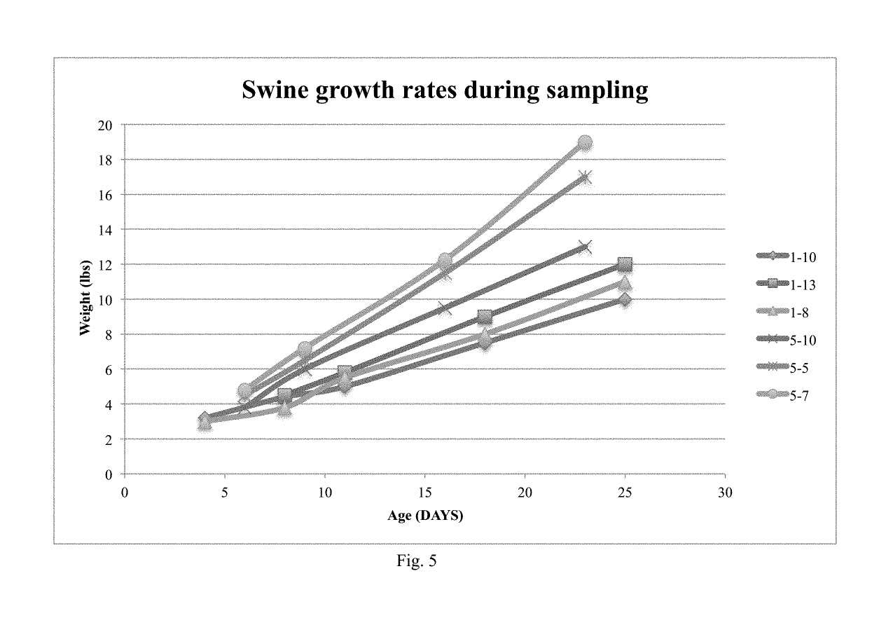

To insure the sampling procedure a series of swine were biopsied, in accordance with the inventive procedure, and weighed with samples starting as early as 1 day of age up to 7 weeks of age. The data is provided in FIGS. 4 and 5 wherein each data point is a separate biopsy as a function of time. In the data presented in FIGS. 4 and 5 the numerical representation for each sample indicates the litter and animal, respectively. By way of example, a numerical representation such as “1-10” would indicate the tenth animal of the first litter with the assigned number having no significance except as a guide to monitor the animals for consistency in analysis. The results presented in FIGS. 4 and 5 demonstrate that the animals gained a similar and healthy amount of weight over the course of sampling thereby indicating minimal or no effects of the sampling procedure causing reduced weight gain. Animals 19-10 and 19-12 were sacrificed at 4 weeks of age.

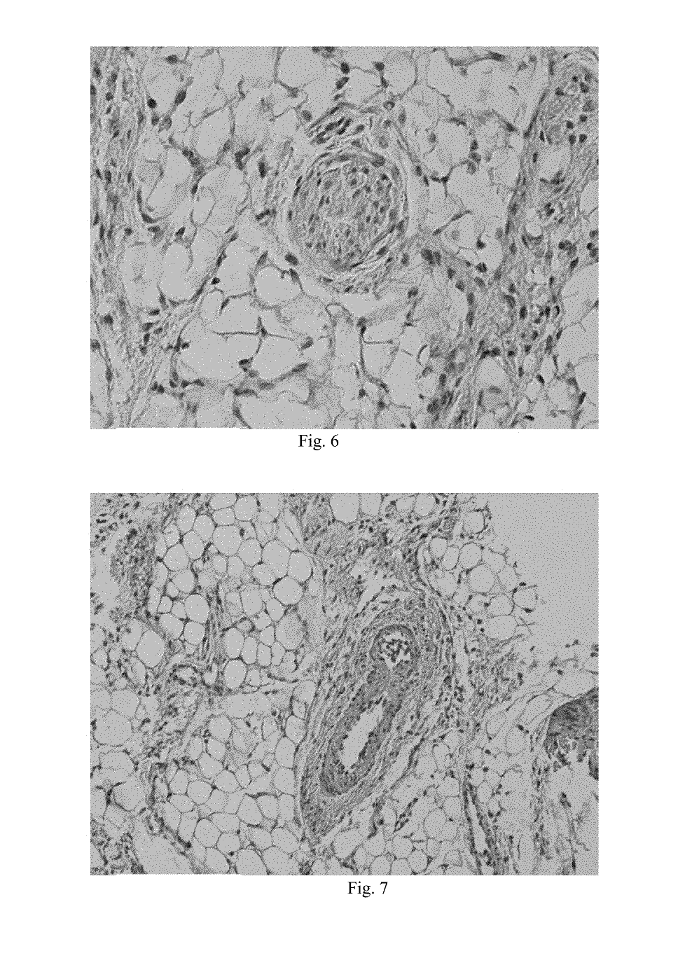

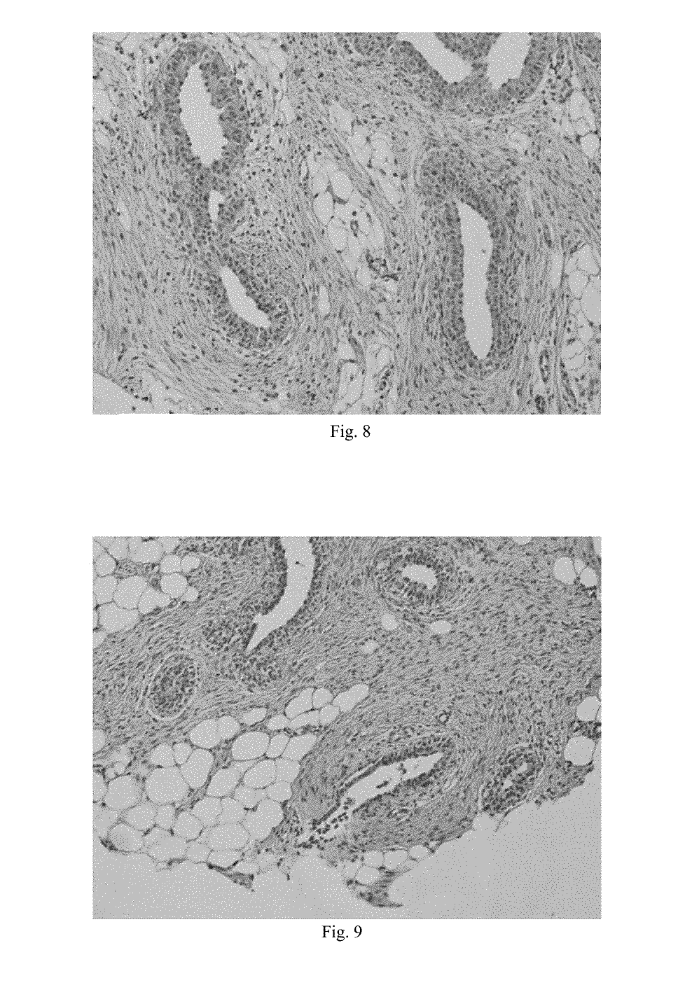

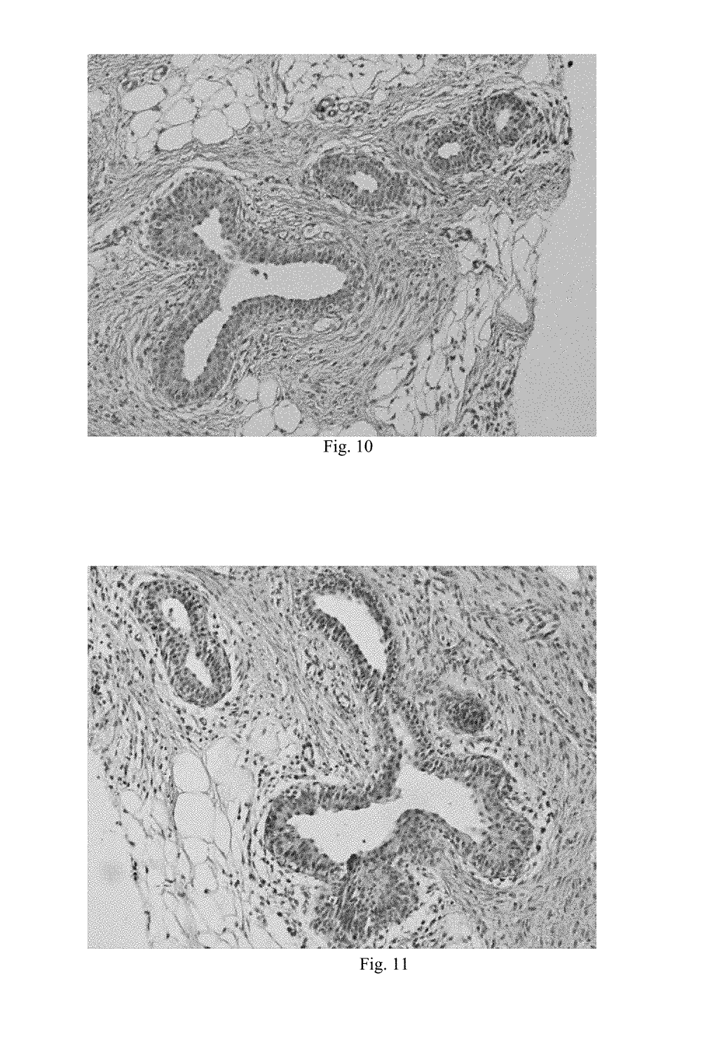

FIGS. 6-11 represent samples taken from the same pig at 4 days, 8 days, 11 days, 18 days, 25 days and 32 days, respectively. The samples demonstrate normal differentiation of mammary tissue over time with no noted biasing or alteration of the tissue differentention. This indicates the unexpected results wherein mammary tissue development, from a single specimen, can be followed at the cellular level without deteriment to the animal or alteration of the differentiation.

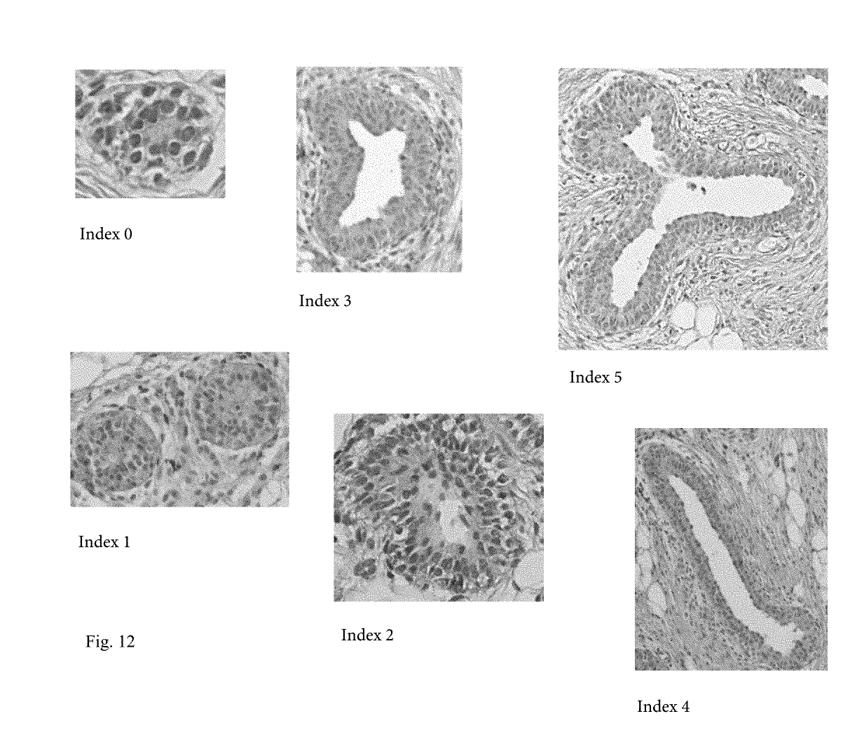

A contingency analysis was conducted for two pigs, with an assigned identity of 19-12 and 20-7, at the age of 4 weeks to confirm that the mammary biopsy procedure produces a tissue sample that is representative of, and no different than, the results obtained by necropsy. In the contingency analysis cells obtained by the inventive technique, and cells obtained by necropsy, were assigned a development index. The development maturity index will be described with reference to FIG. 12. As illustrated in FIG. 12, cells with a maturity index of 0 are a cluster of cells without lumen and 1 ring layer, cells with a maturity index of 1 present as a cluster of cells without lumen and more than 1 ring layer of cells, cells with a maturity index of 2 present as loosely organized cells with lumen, cells with a maturity index of 3 present as semi-organized cells with lumen, cells with a maturity index of 4 present as dense and organized cells with lumen, cells with a maturity index of 5 present as dense and organized cells with lumen and branching. The results obtained by the contingency analysis are provided in Table 1 for pig 19-12 and Table 2 for pig 20-7.

TABLE 1

| Technique | | Index 0 | Index 1 | Index 2 | Index 3 | Index 4 | Index 5 | Total |
|---|---|---|---|---|---|---|---|---|
| Biopsy | Samples | 0 | 2 | 6 | 2 | 0 | 0 | 10 |
| Biopsy | Total % | 0.00 | 3.23 | 9.68 | 3.23 | 0.00 | 0.00 | 16.13 |
| Biopsy | Col. % | | 13.33 | 24.00 | 16.67 | 0.00 | 0.00 | |
| Biopsy | Row % | 0.00 | 20.00 | 60.00 | 20.00 | 0.00 | 0.00 | |
| Necropsy | Samples | 0 | 13 | 19 | 10 | 9 | 1 | 52 |
| Necropsy | Total % | 0.00 | 20.97 | 30.65 | 16.13 | 14.52 | 1.61 | 83.87 |
| Necropsy | Col. % | | 86.67 | 76.00 | 83.33 | 100.00 | 100.00 | |
| Necropsy | Row % | 0.00 | 25.00 | 36.54 | 19.23 | 17.31 | 1.92 | |
| Total | Samples | 0 | 15 | 25 | 12 | 9 | 1 | 62 |
| | Total % | 0.00 | 24.20 | 40.33 | 19.36 | 14.52 | 1.61 | |

The statistical analysis, based on 62 samples, with 4 degrees of freedom, for pig 19-12 resulted in a loglike of 2.3179579 and a R²(U) of 0.0272. A Liklihood Ratio for X² was 4.636 with a Probability>X² of 0.3267. The Pearson analysis for X² was 3.157 with a Probability>X² of 0.5319.

TABLE 2

| Technique | | Index 0 | Index 1 | Index 2 | Index 3 | Index 4 | Index 5 | Total |
|---|---|---|---|---|---|---|---|---|
| Biopsy | Samples | 2 | 9 | 15 | 3 | 2 | 0 | 31 |
| Biopsy | Total % | 3.13 | 14.06 | 23.44 | 4.69 | 3.13 | 0.00 | 48.44 |
| Biopsy | Col. % | 100 | 50 | 48.39 | 42.86 | 40 | 0.00 | |
| Biopsy | Row % | 6.45 | 29.03 | 48.39 | 9.68 | 6.45 | 0.00 | |
| Necropsy | Samples | 0 | 9 | 16 | 4 | 3 | 1 | 33 |
| Necropsy | Total % | 0.00 | 14.06 | 25.00 | 6.25 | 4.69 | 1.56 | 51.56 |
| Necropsy | Col. % | 0.00 | 50.00 | 51.61 | 57.14 | 69.99 | 100.00 | |
| Necropsy | Row % | 0.00 | 27.27 | 48.48 | 12.12 | 9.09 | 3.03 | |
| Total | Samples | 2 | 18 | 31 | 7 | 5 | 1 | 64 |
| | Total % | 3.13 | 28.13 | 48.44 | 10.94 | 7.81 | 1.56 | |

The statistical analysis, based on 64 samples, with 5 degrees of freedom, for pig 20-7 resulted in a loglike of 2.2366694 and a R²(U) of 0.0264. A Liklihood Ratio for X² was 4.473 with a Probability>X² of 0.4835. The Pearson analysis for X² was 3.316 with a Probability>X² of 0.6514.

The contingency analysis tests the null hypothesis which is based on confirmation of a hypothesis that there is no statistical difference in the distribution of the two populations or that the response level rates are the same for the two techniques. A Probability>X² of less than 0.05 indicates that any difference between the biopsy sample of the inventive technique and necropsy sample is due to something other than chance and therefore the methods are different. A Probability>X² of 0.3267, based on the Liklihood Ratio, or 0.5319, based on a Pearson analysis, indicates that any differences in the samples are likely due to chance and therefore the null hypothesis is correct and there is no statistical difference in the responses for the two methods. The inventive technique is therefore considered an equivalent to the results obtained by necropsy.

For purposes of demonstration the sampling of Hereford Beef Cattle, as a representative mammal, will be described. To demonstrate the invention sample, collection can begin when the calves are 1 week of age and continue for 6 weeks of sampling until the animals are 7 weeks old.

The animal being sampled is preferably manually restrained for no longer than 10 minutes each. In one embodiment each animal will be sampled once a week, for 6 weeks with samples collected from the udder of females and the neck region of both males and females. In another embodiment animals are sampled once but the sampling point consist of animals of different ages. Approximately 6 months after biopsies blood samples were taken and weight monitored. In one embodiment it is preferably to collect −5cc of blood for comparison purposes and subsequent study. Body weights are preferably recorded at each date of sampling.

Once the animal being tested has been restrained, the sample site is preferably cleaned such as with a triple wash of chlorhexidine and betadine. A numbing agent, such as Lidocaine (2%) is preferably administered at the sample site via intradermal and subcutaneous routes, and a small tissue sample is then removed from the parenchyma of the mammary gland/udder of females, and/or the neck region of males and females. Sample collection with a 14 gauge needle is suitable for demonstration of the invention. The animal is preferably evaluated for approximately 5 minutes following the procedure to ensure any bleeding has stopped from the biopsy sample site and the blood collection site. If the animal is bleeding it is preferable to apply pressure. After being released from the restraint the animal is allowed to return to standard housing and feeding preferably with daily observations for 3-5 days for any signs of infection. If the animal appears to have an infection appropriate treatment is preferably provided. During this procedure, the animal is preferably closely monitored for any signs of pain.

The present invention provides many advantages in the art. A particular advantage is in the area of sample testing and analysis. The ability to obtain a minimally invasive sample, with minimal or no trauma to the animal, eliminates the necessity of invasive complicated surgeries or necropsy typically required to achieve similar results. Furthermore, necropsy is final and time studies necessarily require a different animal at each time sequence. The present invention allows for studies of tissue development from a single animal as a function of time thereby significantly increasing the information available. This eliminates the correlation of data across multiple animals. The samples obtained with the present invention are very small compared to prior art techniques. Therefore, the storage containers are smaller and more portable which allows for sampling of animals in the field with less movement of the animal. Large samples typically require liquid nitrogen tanks brought to the field, which complicates the sample collection and storage.

Prior art samples are typically much larger than actually needed resulting in the requirement of intermediate processing operations to obtain a sample suitable for insertion into histology cassettes and storage containers. The additional handling and manipulation can introduce contamination and damage which may alter the ultimate results. With the present invention the sample comes into contact with only the biopsy needle and sterile transfer needle without further sample preparation or handling. The reduced handling, with the inventive technique, further minimizes sample exposure to the environment, relative to the prior art, thereby reducing the number of uncontrolled variables in the sample set.

The present invention also provides advantages in sample preparation. Due to the small size, smaller storage containers can be used thereby providing for a stable sample for up to 24 hours at room temperature. Prior art samples need to be frozen in liquid nitrogen and are not stable at room temperature. Therefore, the prior art samples cannot be immediately introduced to RNA stabilization buffer unless very large samples are introduced which is impractical. The samples are also more easily transported from the field or between labs.

The present invention also provides improvements in sample preparation. Smaller samples allow for the use of minimal reagents thereby minimizing the cost. With the prior art the sample either needs to be cut at the field site, which may introduce contaminants, or excess reagents must be used neither of which is desirable. Samples of the present invention are stable in RNA stabilization reagents at −80° C. for 1-2 years at minimum and require minimal storage space whereas prior arts samples require larger freezer space. The present invention includes the collection of smaller samples which are more easily penetrated with formalin and therefore the entire sample is more easily fixed. Penetration in large samples is less efficient often resulting in unfixed portions towards the center. With samples obtained by the inventive techniques, samples for histological analysis can be transferred from formalin to ethanol for long term storage at room temperature.

Sample preparation for analysis is improved for the inventive technique. In the preparation of RNA samples, for example, tissues can be homogenized with small, hand-held tools with sterile and disposable probes. By comparison, samples obtained by prior art techniques require large tools for homogenizing and the larger tools are not typically available with sterile and disposable probes thereby potentially introducing contamination. The samples can be prepared for histology by embedding in much less paraffin than prior art samples.

The samples obtained by the inventive technique allow for sectioning at thicknesses down to 5 microns whereas with larger samples obtained by prior art methods sectioning at 20 microns is often required resulting in overlaid planes of cells. The smaller samples allow for many replicates on a single slide whereas the prior art obtained samples typically only allow 1-2 sections per slide. The smaller samples also allow for a greater paraffin to sample ratio, and therefore easier sectioning, than the prior art obtained samples. The increased ratio of paraffin allows for normal microtome blades to be used whereas stronger blades are typically required for the larger samples. The thinner sections, available with the samples obtained by the inventive technique, adhere more easily to the glass microscope slides whereas the thick sections of the prior art can curl or fold and do not adhere as easily to the slides. The thin samples, such as 5-7 microns, allow for a single plane to be stained wherein thick sections take up excess stain which hinders visualization.

Samples can be prepared for histological procedures using standard laboratory techniques. The samples can be fixed in 10% formalin, neutralized and buffered, for 18 hours. The samples can then be serially dehydrated in alcohol, clarified in xylene, infiltrated with liquid paraffin and embedded in paraffin blocks. The blocks can be cut into slices, mounted on slides, stained with hematoxylin-eosin or other staining reagents and covered with cover slips using conventional mounting mediums.

The following references are incorporated by reference in their entirety: U.S. Pat. No. 4,521,409; U.S. Pat. No. 5,059,586; U.S. Pat. No. 6,723,518; U.S. Pat. No. 5,798,266; U.S. Pat. No. 7,303,740; U.S. Pat. No. 5,846,739; U.S. Pat. No. 5,627,034; U.S. Pat. No. 7,452,727; U.S. Pat. Publ. No. 2005/0013818; U.S. Pat. No. 5,614,364; U.S. Pat. No. 5,041,371; U.S. Pat. No. 8,087,381; U.S. Pat. No. 7,886,691; U.S. Pat. No. 6,610,496; U.S. Pat. No. 8,003,328; U.S. Pat. No. 8,753,889; U.S. Pat. No. 7,123,762; WO 2006074367; U.S. Pat. No. 6,287,254; U.S. Pat. No. 7,029,441 and U.S. Pat. Publ. No. 2006/0271302.

The invention has been described with reference to the preferred embodiments without limit thereto. One of skill in the art would realize additional embodiments and improvements which are not specifically set forth herein but which are within the scope of the invention as more specifically set forth in the claims appended hereto.

Claimed is:

1. A method for predicting manifested attributes of mammalian tissue comprising:
providing a biopsy needle with a bore diameter of at least 0.9081 mm (20 gauge) to less than 2.413 mm (13 gauge) with a specimen notch, of at least 2 mm to no more than 16 mm;
inserting said needle into said mammalian tissue of a pre-pubescent living mammal; and
extracting sample tissue from said mammalian tissue.

2. The method for predicting manifested attributes of mammalian tissue of claim 1 further comprising a second inserting and a second extracting thereby obtaining a second sample tissue.

3. The method for predicting manifested attributes of mammalian tissue of claim 2 wherein said second inserting is at least one day after said inserting.

4. The method for predicting manifested attributes of mammalian tissue of claim 1 wherein said mammalian tissue is asymptomatic tissue.

5. The method for predicting manifested attributes of mammalian tissue of claim 1 wherein said sample tissue is selected from skin, adipose tissue and muscle tissue.

6. The method for predicting manifested attributes of mammalian tissue of claim 1 wherein said sample tissue is selected mammary gland tissue and muscle tissue.

7. The method for predicting manifested attributes of mammalian tissue of claim 6 wherein said sample tissue is not blood or hair.

8. The method for predicting manifested attributes of mammalian tissue of claim 1 wherein said pre-pubescent mammal is no more than 3 months of age.

9. The method for predicting manifested attributes of mammalian tissue of claim 8 wherein said pre-pubescent mammal is less than 2 months of age.

10. The method for predicting manifested attributes of mammalian tissue of claim 9 wherein said pre-pubescent mammal is less than 1 month of age.

11. The method for predicting manifested attributes of mammalian tissue of claim 1 wherein said pre-pubescent mammal is selected from swine, cattle, ovine, caprine, primates, rodent, canine, and feline.

12. The method for predicting manifested attributes of mammalian tissue of claim 1 wherein said biopsy needle is a spring activated biopsy needle.

13. The method for predicting manifested attributes of mammalian tissue of claim 1 wherein said bore diameter is at least 1.270 mm (18 gauge).

14. The method for predicting manifested attributes of mammalian tissue of claim 1 wherein said specimen notch is at least 8 mm.
